(19) United States
(12) Patent Application Publication
(10) Pub. No.: US 2009/0003668 A1
(43) Pub. Date: Jan. 1, 2009

(54) IMAGE PROCESSING METHOD, IMAGE PROCESSING PROGRAM, AND IMAGE PROCESSING DEVICE

(75) Inventor: Kazuhiko MATSUMOTO, Tokyo (JP)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: ZIOSOFT INC., Tokyo (JP)

(21) Appl. No.: 12/030,447

(22) Filed: Feb. 13, 2008

(30) Foreign Application Priority Data
Jun. 29, 2007 (JP) 2007-172781

(51) Int. Cl. A61B 5/00

(52) U.S. Cl. 382/128

(57) ABSTRACT

An image processing method for specifying a region of interest encapsulating without omission a part of interest through a simple operation. Start and end points specified on a blood vessel center curve define the region of interest range of the blood vessel. A front point at the frontmost position and rear point at the rearmost position in the projection direction are determined based on a center axis from the start to end points. A front specified plane located frontward by a predetermined distance from a plane perpendicular to the projection direction and intersecting the front point is specified. A rear specified plane located rearward by a predetermined distance from a plane perpendicular to the projection direction and intersecting the rear point, is specified. A region of interest encapsulating the part of interest, without omission, between the front specified plane and the rear specified plane is defined.

### BACKGROUND OF THE INVENTION

The present invention relates to an image processing method, an image processing program, and an image processing device.

Medical image information of three or more dimensions (volume data), which has been prepared by medical diagnostic image apparatuses such as x-ray CT devices, nuclear magnetic resonance imaging devices (MRI devices) and the like, has conventionally been visualized and used in diagnostics and therapy.

For example, such volume data are known to be used in volume rendering methods, such as multiplanar reconstruction for effectively observing each cross-sectional slice of organs and the like (MPR), maximum intensity projection method for effectively observing blood vessels as three-dimensional displays (MIP). Other volume rendering methods include the raycast method, minimum intensity projection (MinIP), raysum method, average value method and the like.

An observation of region of interest is often extracted in three-dimensional diagnostic imaging. That is, an organ which is an observation object, or the organ and the surrounding region are extracted from the information of the entire body included in the volume data. Accurate extraction of an organ, however, requires high precision processing such. as that disclosed in Japanese Laid-Open Patent Publication No. 2005-185405, and difficult operations are necessary in order to extract the surrounding region in a suitable range. When observing an object that runs in directions such as blood vessels, for example, it is desirable to observe only part of the blood vessel in detail rather than to observe the entire blood vessel. In such a case, slab-MIP is known as a simple and effective display method for observing region of interest as a three-dimensional display.

Slab-MIP is a three-dimensional display method that displays the region between two specified sectional planes using the MIP method. Accordingly, since the unnecessary part of the blood vessel outside the range of the region is eliminated and only the part desired for observation (part of interest) is three-dimensionally displayed, the method is exceptionally effective for detailed observation focusing on the part of interest.

When preparing the slab-MIP, the user must pre-select the two sectional planes (specified planes). The operation by which the user specifies the two sectional planes is accomplished by the following methods.

One method employs a mouse or the like to specify one target point of a coronary blood vessel on the periphery of the heart displayed in a volume rendered image (VR image) displayed on a monitor. Then, a sectional plane passing through the specified point is set as a reference plane, and two sectional planes spaced by a predetermined distance on opposite sides of the reference plane are selected.

Another method employs a mouse or the like to specify two points that include a target part of interest of a coronary blood vessel on the periphery of the heart in a volume rendered image (VR image) displayed on a monitor. Then, cross-sectional planes that respectively pass through these two selected points are determined.

The area between the two specified planes obtained by these methods is set as a region of interest, and this region of interest is provided to a MIP process to generate a MIP image so as to three-dimensionally display, for example, a coronary blood vessel which is present between the two specified planes.

However, the user cannot confirm the depth of the part of interest (distance between the two specified planes), since the user made the specification using a mouse or the like while viewing a volume rendered image displayed on a monitor. There are instances, therefore, when a portion of the part of interest 100 shown in FIG. 1 forwardly or rearwardly extends out from between the two specified planes Sf and Sr (region of interest Z) even though the user set a region of interest between two specified planes.

Thus, a problem arises when a portion of the part of interest 100 extends out and the extending portion is omitted when the MIP image is generated since the region of interest 100 cannot be observed in detail. It is particularly difficult to set a region with tortuous tissue such as a blood vessel.

In such cases, the volume rendered image displayed on the monitor is rotated to confirm the depth of the part of interest and the like, and the thickness is reset so as to include the entirety part of interest. This resetting operation is extremely troublesome since high skill and long experience is required.

Japanese Laid-Open Patent Publication No. 2006-187531 and U.S. Pat. No. 7,170,517 disclose methods for generating MIP images that do not omit parts of interest by dividing a tortuous blood vessel into a plurality of parts along the lengthwise direction, setting a thickness for each of the divided parts, and subjecting the parts to MIP processing.

However, these methods for setting the thickness of each part of a divided blood vessel in MIP processing require extremely long processing calculation times, and require costly image processing devices capable of running at high signal processing speeds. Furthermore, the obtained region of interest has an extremely narrow range along the lengthwise direction of the blood vessel since the blood vessel is divided into a plurality of parts in the lengthwise direction and a thickness is set for each of the divided parts. Although an image of the part of interest of the blood vessel is displayed, an image of organs or the like in the vicinity of the part of interest of the blood vessel are not displayed. This makes it difficult to confirm in detail the part of interest of the blood vessel while grasping the relative relationship of the part of interest of the blood vessel blood vessel to organs in the vicinity of the part of interest.

### SUMMARY OF THE INVENTION

The present invention provides an image processing method, an image processing program, and an image processing device for specifying a region of interest encapsulating, without omitting, a part of interest through a simple operation.

One aspect of the present invention is a method for generating an image by projecting image data of three or more dimensions in a region of interest on a two-dimensional plane. The method includes setting a guide curve, setting a reference direction, displaying the guide curve on a screen of a monitor, specifying two or more specification points on the guide curve to designate a partial curve of the guide curve, acquiring a front point located on the partial curve at a rearmost position in the reference direction, acquiring a rear point located on the partial curve at a frontmost position in the reference direction, specifying two planes that encapsulate the partial curve viewed from the reference direction based on the front point and the rear point, and defining the region of interest based on the two specified planes.

Another aspect of the present invention is a computer program device including a computer readable recording medium encoded with a program for projecting, on a two-dimensional plane, image data of three or more dimensions in a region of interest and generating an image by executing either one of independent processing or distributed processing with at least one computer. The program when executed by the at least one computer performing a method including setting a guide curve, setting a reference direction, displaying the guide curve on a screen of a monitor, specifying two or more specification points on the guide curve to designate a partial curve of the guide curve, acquiring a front point located on the partial curve at a frontmost position in the reference direction, acquiring a rear point located on the partial curve at a rearmost position in the reference direction, specifying two planes that encapsulate the partial curve viewed from the reference direction based on the front point and the rear point, and defining the region of interest based on the two specified planes.

A further aspect of the present invention is a device for projecting, on a two-dimensional plane, image data of three or more dimensions in a region of interest and generating an image by executing either one of independent processing or distributed processing with at least one computer. The device including a means for setting a guide curve, a means for setting a reference direction, a means for displaying the guide curve on a screen of a monitor, a means for specifying two or more specification points on the guide curve to designate a partial curve of the guide curve, a means for acquiring a front point located on the partial curve at a frontmost position in the reference direction, a means for acquiring a rear point located on the partial curve at a rearmost position in the reference direction, a means for specifying two planes that encapsulate the partial curve viewed from the reference direction based on the front point and the rear point, and a means for defining the region of interest based on the two specified planes.

Other aspects and advantages of the present invention will become apparent from the following description, taken in conjunction with the accompanying drawings, illustrating by way of example the principles of the invention.

### BRIEF DESCRIPTION OF THE DRAWINGS

The invention, together with objects and advantages thereof, may best be understood by reference to the following description of the presently preferred embodiments together with the accompanying drawings in which:

FIG. 1 is a diagram showing a conventional method for specifying two sectional planes;

FIG. 2 is a schematic diagram showing an image display apparatus according to a preferred embodiment of the present invention;

FIG. 3 is a schematic block diagram of the image display apparatus in the preferred embodiment;

FIG. 4 is a diagram illustrating a volume rendered image;

FIG. 5 is a diagram illustrating the method for specifying the start point and end point of the part of interest from the volume rendered image;

FIG. 6 is a diagram illustrating a MIP image;

FIG. 7 is a diagram illustrating a specified plane defining a region of interest that encapsulates a part of interest;

FIG. 8 is a diagram illustrating a MIP image;

FIG. 9 is a diagram illustrating a specified plane obtained from the start point and end point;

FIG. 10 is a diagram showing the MIP values of one pixel;

FIG. 11 is a flowchart illustrating the image process of the present invention; and

FIG. 12 illustrates a modification of the specification of specification points of a part of interest of the present invention.

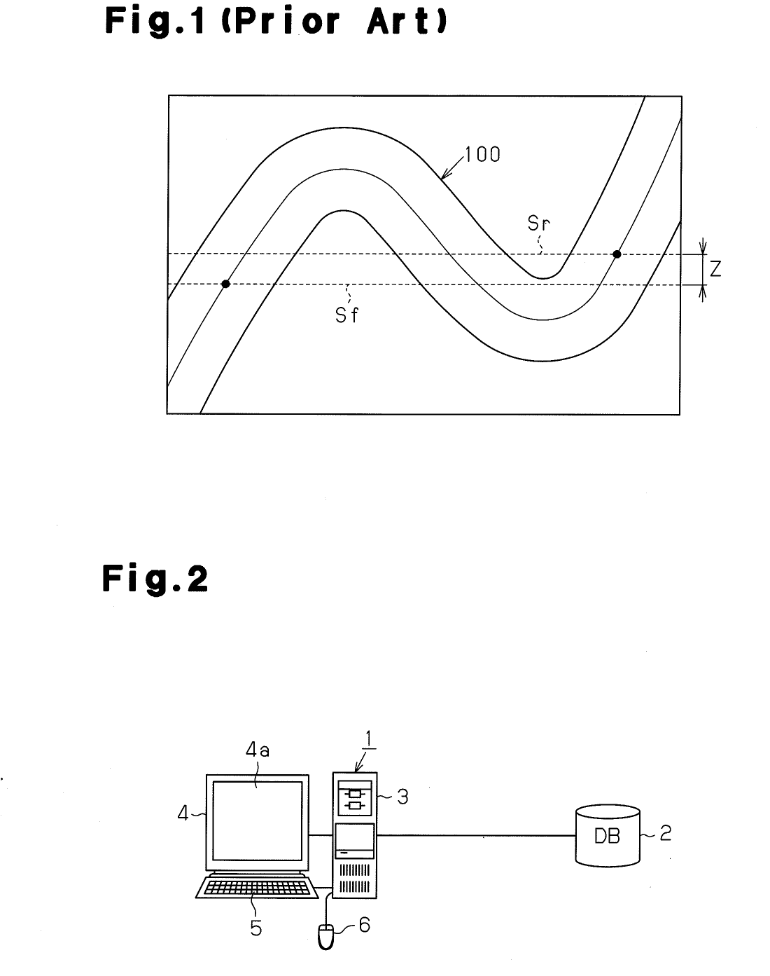

### DETAILED DESCRIPTION OF THE PREFERRED EMBODIMENTS

An image processing device according to a preferred embodiment of the present invention will now be discussed with reference to FIGS. 2 through 11.

As shown in FIG. 2, an image display device 1 reads, for example, CT (computerized tomography) image data acquired by a CT scanner from a database 2, generates each type of medical diagnostic image from the CT image data, and displays these images on a monitor 4. The embodiment is not limited to CT scanner, for example, medical image processing devices such as MRI (magnetic resonance imaging) and the like, or combinations of such image processing devices are acceptable.

The image display device 1 is provided with a computer (computer, workstation, personal computer) 3, monitor 4, and input devices such as a keyboard 5 and mouse 6 or the like. The computer 3 is connected to a database 2.

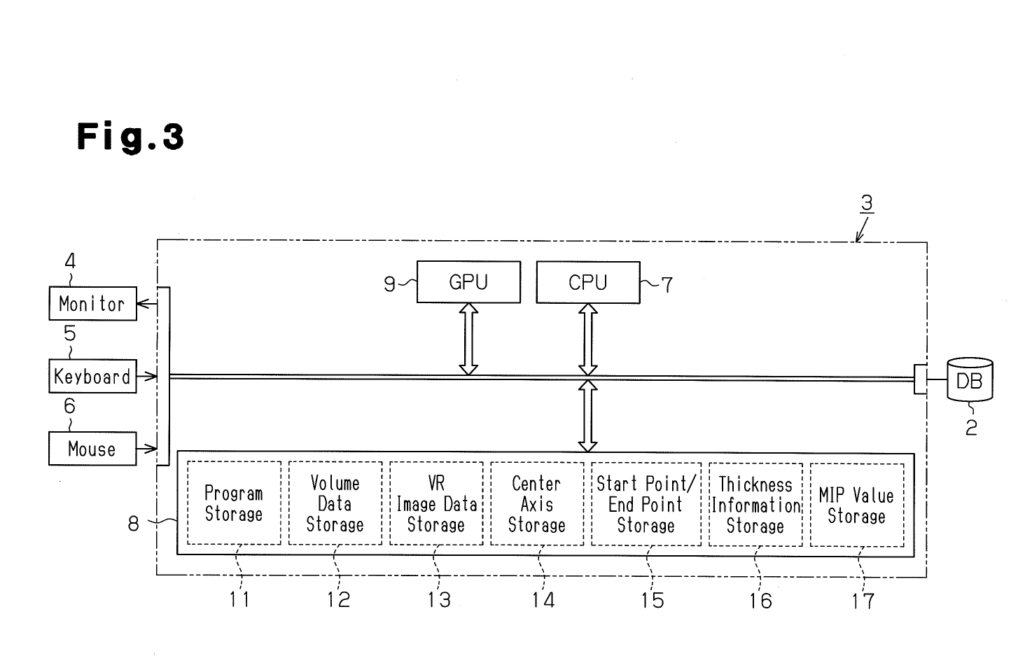

FIG. 3 is a schematic block diagram of the image display device 1. The computer 3 includes a central processing unit (CPU) 7 functioning as an image processing device, a memory 8 configured by a hard disk or the like, and a graphic processing unit (GPU) 9.

The memory 8 includes a program storage 11, volume data storage 12, VR image data storage 13, center axis storage 14, start-point/end-point storage 15, thickness information storage 16, and MIP value storage 17.

The program storage 11 stores programs (application programs) for executing image processing.

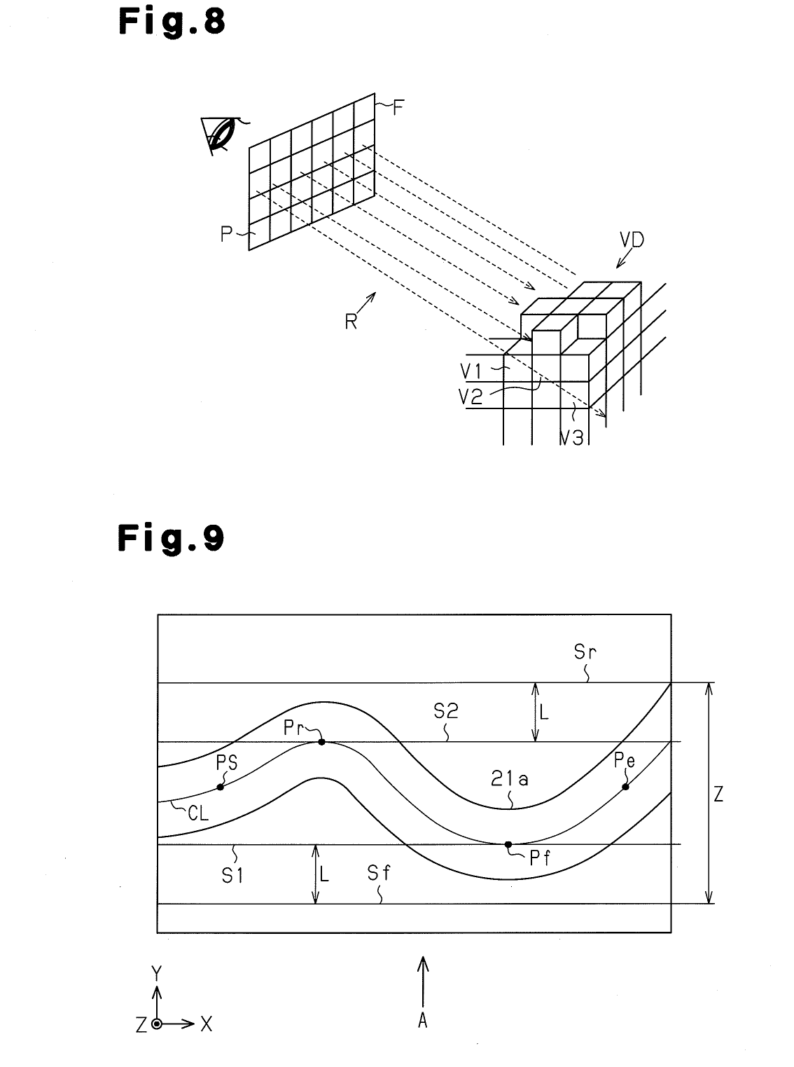

The volume data storage 12 temporarily stores the volume data VD (refer to FIG. 8), which is obtained from the CT image data read from the database 2 or a hard disk.

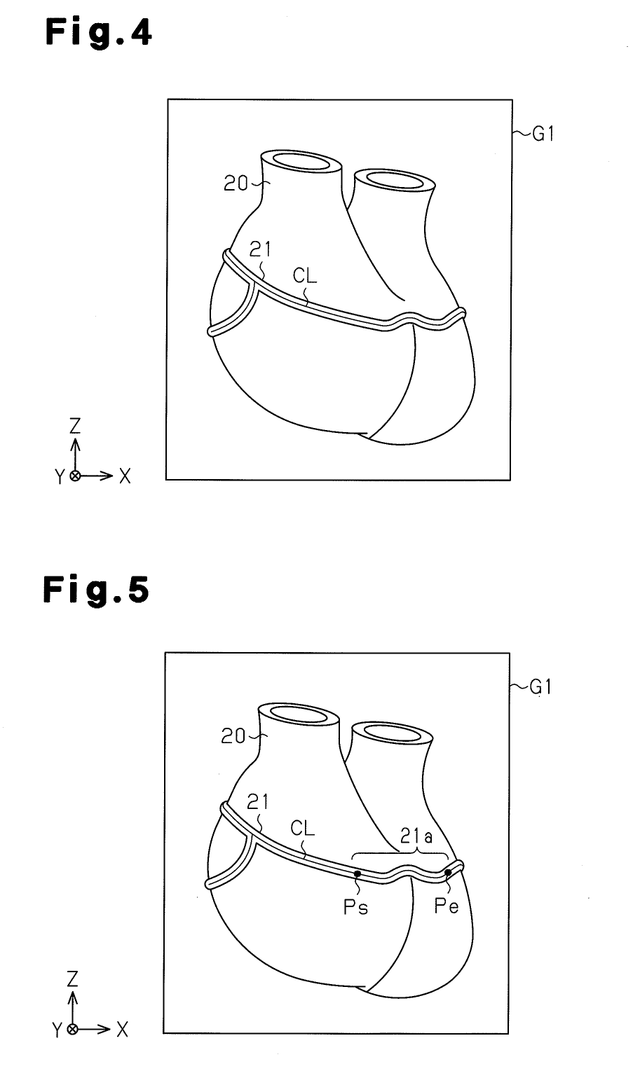

The VR image data storage 13 temporarily stores the data of a volume rendered image G1 displayed on the monitor 4 as shown in FIG. 4 when volume rendering process that uses volume data VD stored in the volume data storage 12 is executed. The volume rendered image G1 shown in FIG. 4 displays a heart 20 as an organ, and a blood vessel 21 as vascular tissue in the vicinity of the heart 20.

The center axis storage 14 stores data of the center axis CL as a guide curve of the blood vessel 21 which curves in three-dimensional directions in the volume rendered image G1 displayed on the monitor 4. The data of the center axis CL are used to draw a guide curve overlaid on the blood vessel 21. The center axis data is three-dimensional curvature data determined by well known methods such as that disclosed in, for example, Japanese Laid-Open Patent Publication No. 2004-358001.

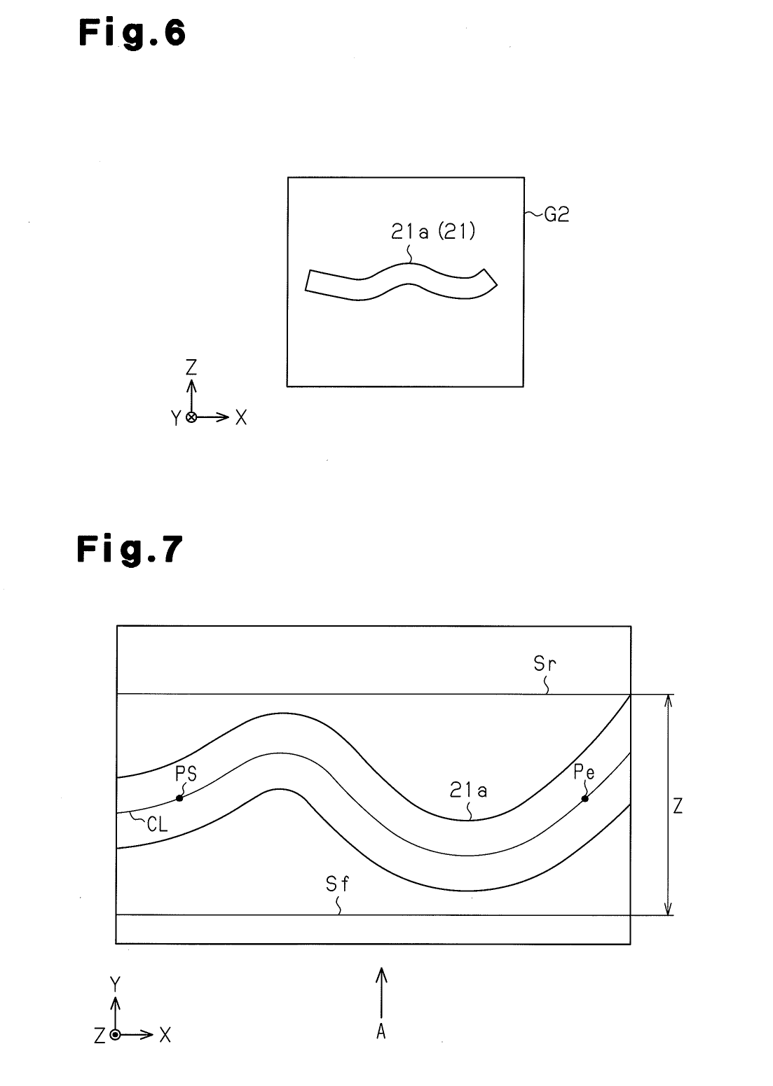

The start-point/end-point storage 15 stores the range (start point Ps and end point Pe) of the part of interest 21a of the blood vessel 21 included in the volume rendered image G1, as shown in FIG. 5. The range (start point Ps and end point Pe) 21a is used to obtain a MIP image G2 as an image of interest shown in FIG. 6 by a MIP (maximum intensity projection) process using the volume data VD. In the present embodiment, position information which includes the three-dimensional coordinates of the start point Ps and the end point Pe is obtained by the user who clicks the mouse 6 to specify the start point Ps and end point Pe. The start point Ps and end point Pe are two specification points that specify the range of the part of interest 21a of the blood vessel 21 in the volume rendered image G1 displayed on the monitor 4, as shown in FIG. 5, and store the position information.

The thickness information storage 16 temporarily stores the data for specifying the range for MIP processing when obtaining the MIP image G2 by MIP processing using the volume data VD. The data specifying the range includes the position information of the start point Ps and the end point Pe, and the front specified plane Sf and rear specified plane Sr obtained using the three-dimensional curve data of the center axis CL. As shown in FIG. 7, the front specified plane Sf and rear specified plane Sr are two specified planes that determine a region of interest Z (thickness of region) encapsulating the part of interest 21a of the blood vessel 21 viewed from the projection direction A, which serves as a reference direction.

The MIP value storage 17 stores the MIP values of all pixels of two-dimensional images used when obtaining the MIP image G2. MIP values are given from a region that includes the part of interest 21a of the blood vessel 21 contained in the volume rendered image G2, by a MIP process using the volume data VD of the region (region of interest Z) between the two specified planes Sf and Sr stored in the thickness information storage 16.

The CPU 7 specifies a region of interest Z from the volume data VD obtained from the CT image data from the database 2 and executes image processing to generate a MIP image G2, by executing programs stored in the program storage 11 of the memory 8. That is, in the present embodiment, the CPU 7 (computer 3) functions as an image processing device by executing image processing programs (guide curve display step, specification point specification step, front point acquisition step, rear point acquisition step, region of interest specifying step, MIP image generation step).

The CPU 7 (computer 3) functions as a guide curve display means, a reference direction setting means, a specification point specifying means, a front point obtaining means, rear point obtaining means, and region of interest specifying means (provisional reference plane preparing means, front specified plane preparing means, rear specified plane preparing means).

The volume data VD is a set of voxels which are elements in three or more dimensions, and the element values are allocated as voxel values at three-dimensional lattice points. In the present embodiment, for example, the voxel data correspond to the value of CT image data, that is, the CT values are voxel values.

The CT image data is obtained by acquiring slice image of human body. Although the image data of a single slice is a two-dimensional slice image of bone, blood vessel, organ or the like, the entirety of the slice image data can be said to be three-dimensional image data since images of a plurality of adjacent slices are obtained. Therefore, CT image data shall henceforth refer to three-dimensional image data that include a plurality of slices.

The CT image data has different CT values for each type of tissue (bone, blood vessel, organ and the like) as a subject of imaging. The CT values are tissue x-ray absorption coefficients using water as a reference, and the type of tissue and type of diseased tissue can be determined from the CT value.

The CT image data includes the coordinate data of slice screens (slice images) of a human body acquired through a CT scan performed by a CT imaging device, and volume data VD include coordinate data and CT values (hereinafter referred to as voxel values).

In the present embodiment, the CPU 7 executes a volume rendered image generating process using the volume data VD to generate a volume rendered image G1 as shown in FIG. 4 and stores the data of the volume rendered image G1 in the VR image data storage 13 of the memory 8. Then, the CPU 7 displays the volume rendered image G1 on the monitor 4 based on the data of the volume rendered image G1 stored in the VR image data storage 13 of the memory 8.

Since the volume rendered image G1 can be generated using well known methods such as MIP, raycasting and the like, details of this image generation are omitted.

The CPU 7 specifies a region of interest Z from the volume rendered image G1 displayed on the screen 4a of the monitor 4, subjects the region of interest Z to MIP (maximum intensity projection) processing, and then displays the resulting MIP image G2 on the screen 4a of the monitor 4 as an image of interest which is shown in FIG. 6.

The region of interest Z is a region defined between the front specified plane Sf and the rear specified plane Sr shown in FIG. 7, and the region of interest Z encapsulates, the range desired for observation (part of interest 21a) of the blood vessel 21 displayed between the two specified planes Sf and Sr, insight of the projection direction A.

The region of interest Z is generated by a region of interest specifying process during the image processing. The region of interest Z, is then subjected to MIP processing to prepare data for the MIP image G2 used to observe the part of interest 21a of the blood vessel 21 within the region of interest Z, and the MIP image G2 (refer to FIG. 6) is displayed on the screen 4a of the monitor 4.

Specifically, in the image processing, a region of interest Z having a predetermined thickness is specified from the volume rendered image G1 (volume data VD), and a MIP image G2 of the part of interest 21a within the region of interest Z is obtained.

The user can specify any two points on the center axis CL by clicking the mouse 6 on the volume rendered image G1 shown in FIG. 4 displayed on the screen 4a of the monitor 4. The points is based on the three dimensional coordinates of a voxel which constitute a volume rendered image GC (volume data VD).

The center axis CL of the blood vessel 21 is displayed overlaid on the blood vessel 21 in the volume rendered image G1 on the screen 4a of the monitor 4 when the CPU 7 executes the guide curve display process, where the center axis CL is generated based on the center axis data stored in the center axis storage 14.

As shown in FIG. 5, the user clicks the mouse 6 to designate two points (start point Ps and end point Pe) of the center axis CL displayed as an overlay on the blood vessel 21. The range of the part of interest 21a of the blood vessel 21, which is included in the volume rendered image G1, is specified by start point Ps and end point Pe. The CPU 7 stores the two points (start point Ps and end point Pe) with three-dimensional coordinates specified by the clicks of the mouse 6 in the start-point/end-point storage 15.

When the start point Ps and end point Pe of the center axis CL are specified, the CPU 7 determines a front point Pf, which is located at the frontmost position, and the rear point Pr, which is located at the rearmost position viewed from viewpoint of projection direction A (line of sight direction) based on the center axis CL from the start point Ps and end point Pe, as shown in FIG. 9 (front point and rear point acquisition process). This determination of points is necessary because the depth of the center axis CL cannot be confirmed from the screen 4a since an image displayed on the screen 4a of the monitor 4 is two-dimensional.

The CPU 7 determines the front point Pf, which is located at the frontmost position, and the rear point Pr, which is located at the rearmost position as shown in FIG. 9 from the center axis data of the three-dimensional curve data and the two points of three-dimensional coordinates (start point Ps and end point Pe). Then, the CPU 7 determines a first plane Si perpendicular to the projection direction A and intersecting the front point Pf positioned nearest in the foreground, and a second plane S2 perpendicular to the projection direction A and intersecting the rear point Pr, which is located at the rearmost position (provisional reference plane generation process).

Next, the CPU 7 determines a plane that is located frontward by a predetermined distance L from the first plane S1 and parallel to the first plane S1 which intersects the front point Pf located at the frontmost position (front specified plane Sf), and a plane that is located rearward by a predetermined distance L from the second plane S2 and parallel to the second plane S2 which intersects the rear point Pr located at the rearmost position (front and rear specified plane generation process). The area between the front specified plane Sf and the rear specified plane Sr is defined as the region of interest Z, and this region of interest Z encapsulates, without omission, the part of interest 21a of the blood vessel 21. The CPU 7 stores the front specified plane Sf and the rear specified plane Sr which define the region of interest Z in the thickness information storage 16. The distance L is preferably set, for example, at a value somewhat greater than the diameter of the blood vessel 21. The distance L may also be set by dynamically acquiring the diameter of the blood vessel 21 at the front point Pf and rear point Pr.

The CPU 7 subjects the region of interest Z generated by the region of interest specification process to MIP processing to generate data of the MIP image G2 used to observe the part of interest 21a within the region of interest Z. Then, the CPU 7 displays the MIP image G2 on the screen 4a of the monitor 4 based on the data of the MIP image G2.

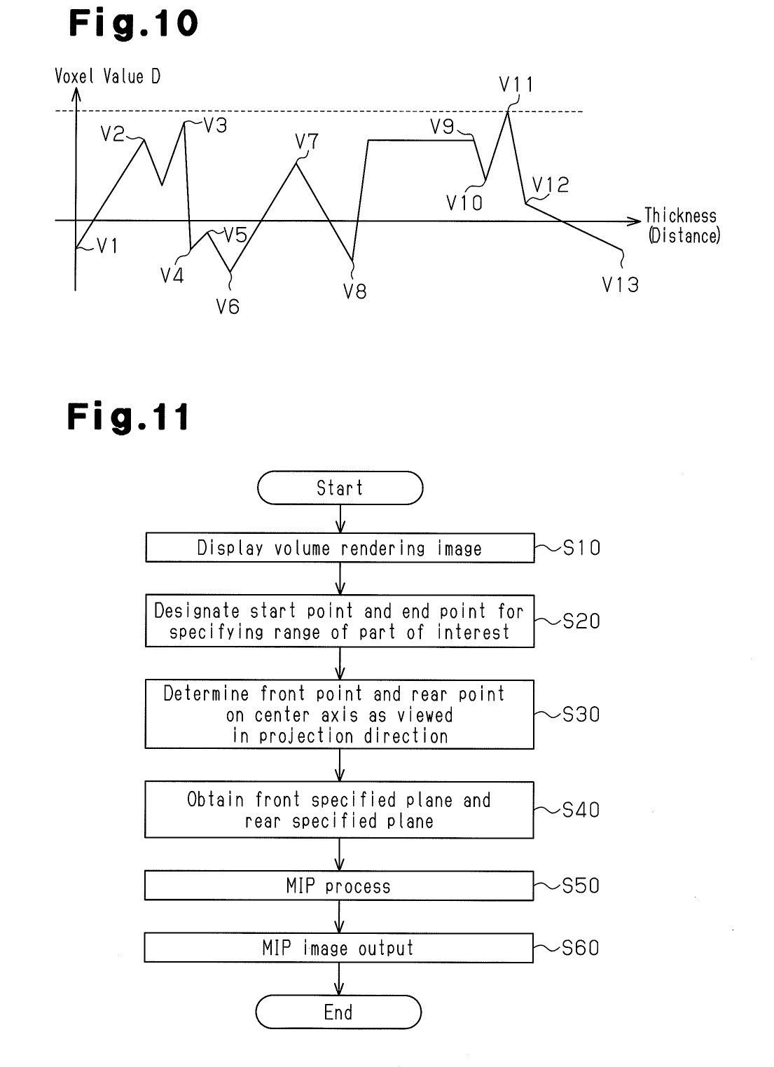

FIG. 10 illustrates the process for generating the MIP image G2 by MIP processing.

MIP (Maximum Intensity Projection method) is one method for converting three-dimensional image data to two-dimensional image data. In the case of parallel projection, for example, paralleled imaginary rays R radiates from a line of sight direction on volume data VD which are the object of observation for each pixel P of a two-dimensional plane F, as shown in FIG. 8. Then, the maximum values for each rays (hereinafter referred to as MIP values) among the voxel values D1, D2, . . . Dn of N individual voxels V1, V2, . . . Vn present on the imaginary rays R are used as two-dimensional image data.

In projection, different two-dimensional image data is projected depending on the direction of the imaginary ray R even though the same volume data VD is the object of observation.

Furthermore, an image of inside of a tubular organ such as a blood vessel or the like can be obtained, for example, with an endoscopic view by perspective projection. This can be done by radiating imaginary rays R radially toward volume data VD from a single particular viewpoint as in direct optical projection methods.

Moreover, an exfoliated image can be obtained, for example, of the inside of tubular tissue (for example, blood vessel 21, trachea, alimentary canal and the like) by radiating imaginary rays R radially in a cylinder and using the volume data VD from viewpoints distributed on a center axis relative to a cylindrical plane disposed around the volume data VD as in cylindrical projection methods. Parralel projection is used as the most suitable for observation of three-dimensional image data in the present embodiment.

When the destination position of the imaginary ray R is not on the lattice, the voxel value D at that position is calculated by performing an interpolation process using the voxel values D of the surrounding voxels V on the lattice.

Specifically, the voxel values D1 to Dn of the voxels V1 to Vn of a single pixel can be expressed, for example, as shown in FIG. 10. FIG. 10 expresses the voxel values D of the voxels V through which the imaginary ray R passes when a single imaginary ray R radiates from each pixel in the line of sight direction, and shows the voxel values D corresponding to the single imaginary ray R shown in FIG. 8. The depth (distance) of the voxel V is plotted on the horizontal axis, and the voxel value D is plotted on the vertical axis in the graph of FIG. 10. As shown in FIG. 10, with regard to a specific pixel Pn, since thirteen individual voxels V1 through V13 are present on the imaginary ray R, and the voxel value D11 of the voxel V11 is the maximum value among these, the voxel value D11 is set as the MIP value of the pixel Pn and stored in the MIP value storage 17.

In this manner, the MIP image G2 shown in FIG. 6, which was obtained by performing MIP processing on the region of interest Z defined based on the start point Ps and end point Pe specified on the volume rendered image G1, can be displayed alongside the volume rendered image G1 on the screen 4a of the monitor 4 by MIP processing the region of interest Z.

As shown in FIG. 3, the computer 3 is provided with a graphic processing unit (GPU) 9. The GPU 9 is a graphic controller chip that supports a high speed three-dimensional graphics function, and is capable of executing drawing process based on programs provided by the user at high-speed. In the present embodiment, post processing is executed by the GPU 9. Therefore, only a short time is required to display the MIP image G2.

Post processing includes processes for color, contrast, and brightness correction to display the calculated MIP image G2 on an output device such as the monitor 4.

Specifically, since the output of many medical imaging devices (CT image, MRT image and the like) are twelve-bit gradient data, the MIP image G2 calculated in the MIP process (MIP values stored in the MIP value storage 17) is also twelve-bit gradient data. However, the monitor 4 of the computer 3 and the like often displays images in which RGB colors are expressed by eight-bit data. Therefore, WL conversion (window width/window level transformation) and LUT conversion (color look-up table transformation) are performed.

Affine transformation is performed to match the size of the screen and forms an image corresponding to the monitor 4.

The image processing performed by the image display device 1 (computer 3) is described below.

FIG. 11 shows a flowchart of the image processing. The user first operates the keyboard 5 and mouse 6 to display a volume rendered image G1 on the screen 4a of the monitor 4 (step S10: guide curve setting step and reference direction setting step). The volume rendered image G1 includes the heart 20 and the blood vessel 21 in the vicinity of the heart 20. This time, the CPU 7 displays the center axis CL of the blood vessel 21 overlaid on the blood vessel 21 in the volume rendered image G1. The center axis CL of the blood vessel 21 is acquired beforehand.

As shown in FIG. 5, a user clicks the mouse 6 on two points (start point Ps and end point Pe) of the center axis CL displayed as an overlay on the blood vessel 21 to designate the start point Ps and the end point Pe. The start point Ps and the end point Pe are represented by three-dimensional coordinates and specify the range of the part of interest 21a of the blood vessel 21 included in the volume rendered image G1 (step S20: specification point designation step). When the start point Ps and end point Pe are specified by the mouse 6, the CPU 7 stores the position information of the start point Ps and end point Pe in the start-point/end-point storage 15.

When the start point Ps and end point Pe on the center axis CL are stored, the CPU 7 determines the front point Pf, which is located at the frontmost position, and the rear point Pr, which is located at the rearmost position, viewed from viewpoint of projection direction A, based on the center axis CL from the start point Ps and end point Pe, as shown in FIG. 9 (step 30: front point and rear point acquisition process). The CPU 7 determines the front point Pf and rear point Pr on the center axis CL from the start point Ps and end point Pe from the center axis data formed by the three-dimensional curve data of the center axis CL and the position information formed by the three-dimensional coordinates of the start point Ps and end point Pe.

Next, the CPU 7 determines the front specified plane Sf and the rear specified plane Sr from the front point Pf and the rear point Pr (step S40: region of interest specifying step, (provisional reference plane generation step, front specified plane generation step, rear specified plane generation step)). Specifically, the CPU 7 first determines a first plane S1 perpendicular to the projection direction A and intersecting the front point Pf, and determines a front specified plane Sf frontward from the first plane S by a predetermined distance L, then stores the front specified plane Sf in the thickness information storage 16, as shown in FIG. 9. The distance L is set so as to position the front specified plane Sf frontward from the anterior external surface of the blood vessel 21 intersecting at the front point Pf.

The CPU 7 determines a second plane S2 perpendicular to the projection direction A and intersecting the rear point Pr, and determines a rear specified plane Sr rearward from the second plane S2 in the background by a predetermined distance L, then stores the rear specified plane Sr in the thickness information storage 16. The distance L is set so as to position the rear specified plane Sr rearward from the posterior external surface of the blood vessel 21 intersecting at the front point Pr.

The region of interest Z which encapsulates without omission the volume data VD of the part of interest 21a of the blood vessel 21 is defined as the region between the front specified plane Sf and the rear specified plane Sr stored in the thickness information storage 16.

The CPU 7 then performs MIP processing of the region of interest Z defined by the front specified plane Sf and the rear specified plane Sr (step S50: MIP image generating process), and the GPU performs the post processing of MIP process to generate a MIP image G2 for observing the part of interest 21a within the region of interest Z. The MIP image G2 which includes the part of interest 21a is then displayed together with the volume rendered image G1 on the screen 4a of the monitor 4 (step S60).

At this time, the volume rendered image G1 which includes the start point Ps and end point Pe is displayed on the screen 4a alongside the MIP image G2. Therefore, the user can easily determine to which part on the volume rendered image G1 the MIP image G2 corresponds. The MIP image G2 may also be displayed alone on the screen 4a.

In the present invention, therefore, the user can accurately specify the region of interest Z that includes the desired part of interest 21a to display by simply clicking the mouse 6 on the center axis CL of the blood vessel 21 on the volume rendered image G1.

The embodiment of the image display device 1 of the present invention has the advantages described below.

(1) A user can accurately specify a region of interest Z which encapsulates without omission the part of interest 21a by a simple operation of clicking the mouse 6 on the range of the part of interest 21a of the blood vessel 21 in the volume rendered image G1.

Therefore, a MIP image G2 of the part of interest 21a can be displayed on the monitor 4 without partial omission through a simple operation.

(2) When the center axis CL of the blood vessel 21 is overlaid on the volume rendered image G1, the user specifies the range of the part of interest 21a by clicking the mouse 6 on two points of the center axis CL (start point Ps and end point Pe) The range of the part of interest 21a can therefore be accurately specified through a simple operation.

Although a user sets two planes (specified planes), which encapsulate a part of interest 21a to obtain a MIP image G2 of the part of interest 21a, perpendicular to the projection direction A in the conventional art, the present invention allows a user to determine two specified planes Sf and Sr which are perpendicular to the projection direction A and encapsulate the part of interest 21a by simply specifying a start point Ps and end point Pe of the part of interest 21a. Therefore, a MIP image G2 of the part of interest 21a can be accurately displayed without omission through a simple operation, without the requirement of a high skills or experience.

(3) The center axis CL displayed overlaid on the blood vessel 21 is three-dimensional curve data, and the specified start point Ps and end point Pe are three-dimensional coordinate values on the center axis CL. Therefore, only a short time is needed to determine the front point Pf, which is located at the frontmost position, and the rear point Pr, which is located in the rearmost position, from viewpoint.

Moreover, the three-dimensional coordinates of the start point Ps and end point Pe on the center axis CL do not change even when the projection direction A changes. Thus, a new front point Pf and rear point Pr corresponding to the change of the projection direction A can be quickly and easily determined using the coordinate values of the start point Ps and end point Pe stored in the start-point/end-point storage 15 and the three-dimensional curve data of the center axis CL stored in the center axis storage 14. As a result, new specified planes Sf and Sr (region of interest S) can be quickly determined in accordance with the change in the projection direction A, and a new MIP image G2 can be displayed in real time.

(4) The front specified plane Sf is frontward by a predetermined distance L from the first plane S1, which is perpendicular to the projection direction A, and intersects the front point Pf. The rear specified plane Sr is rearward by a predetermined distance L from the second plane S2, which is perpendicular to the projection direction A, and intersects the rear point Pr. Therefore, the part of interest 21a of the blood vessel 21 can be accurately encapsulated without omission in the region of interest Z between the front specified plane SF and rear specified plane Sr. Moreover, the image data of the heart 20 and the part of interest 21a in the region of interest Z can be displayed together since the image data used in the MIP process (volume data VD) includes the heart 20 and the like. The positional relationship between the part of interest 21a and the image data of the heart and the like can therefore be easily grasped.

It should be apparent to those skilled in the art that the present invention may be embodied in many other specific forms without departing from the spirit or scope of the invention. Particularly, it should be understood that the present invention may be embodied in the following forms.

(1) At least one among the process for displaying a guide curve in the image processing, process for specifying specification points, process for acquiring a front point, process for acquiring a rear point, process for specifying a region of interest, and process for generating a MIP image may be performed by a plurality of computers.

In a network within a hospital, for example, at least one process may be performed by a plurality of workstations through distributed processing. Thus, large amount of data can be processed, the processing speed can be improved, and the MIP image G2 can be displayed in real time.

(2) Three-dimensional data in the region of interest Z may also be projected on a two-dimensional plane by methods other than MIP. For example, an image of interest may be obtained using a volume rendering method such as MinIP (minimum intensity projection) for projecting minimum values of voxel data D of the voxels V through which the imaginary ray R passes on a two-dimensional plane F, addition method, average value method or the like. MPR (Multi Planer Reconstruction) method with thickness is referred to as one kind of method for displaying an image in a particular region of fixed thickness as an optional slice image equivalent to an MPR image. The MPR method with thickness, however, is included in the present invention since the MPR actually generates a region interposed between two planes using the MIP method, average value method or the like.

(3) The start point Ps and end point Pe may be determined by a single click action of a user, rather than taking two points that clicked by a user as a start point Ps and an end point Pe. The CPU 7 may automatically determine two points (start point Ps and end point Pe) through a calculation using the point clicked by the mouse 6 as a reference point. For example, two points on the center axis CL separated by a predetermined distance in the upstream direction and downstream direction of the blood vessel 21 may be set as the start point Ps and end point Pe using the point clicked by the mouse 6 as a reference point. Furthermore, the point clicked by the mouse 6 may be set as, for example, the start point Ps, and a point on the center axis CL spaced by a predetermined distance from the start point Ps may be set as the end point Pe. Furthermore, two points of, for example, a bifurcated blood vessel 21 in the downstream direction and upstream direction of the blood vessel 21 may be set as the start point Ps and end point Pe using the point clicked by the mouse 6 as a reference point. Thus, the range of the part of interest 21a can be set by an easier operation of a single mouse click.

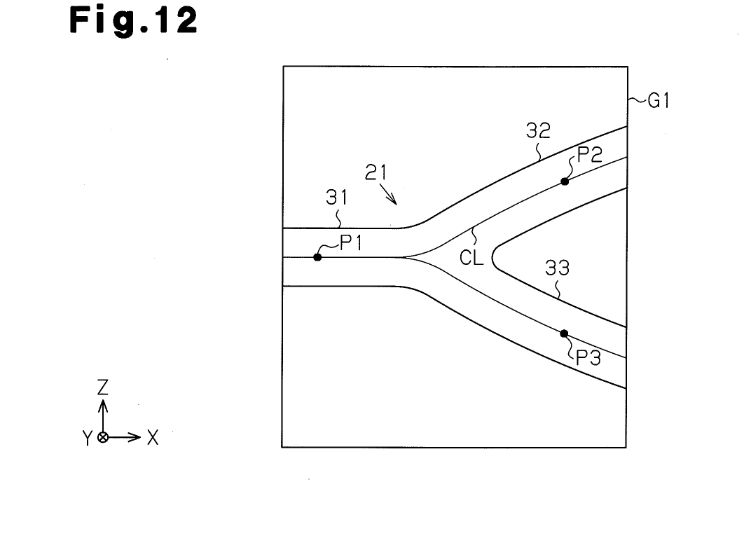

(4) The present invention is also applicable to a middle area of a branched part of interest. In this case, the user specifies end points P1, P2, and P3 on branches 31, 32, and 33 of a blood vessel 21 by clicking the mouse 6, as shown in FIG. 12. Then, specified planes Sf and SR which define the region of interest Z can be determined by determining the front point Pf, which is located at the frontmost position, and a rear point Pr, which is located at the rearmost position, from viewpoint.

(5) The center axis CL of the blood vessel 21 serves as a guide curve need not be displayed as a solid line on the screen inasmuch as the center axis CL may also be displayed as a dashed line, single-dot line, double-dot line and the like. The color of the center axis CL may also be changed as required.

(6) The term of center axis of tubular tissue used in the present specification specifies a tortuous curve along tubular tissue, and is not limited to a center axis strictly connected to the center of gravity of cross-sectionals of tubular tissue. Furthermore, the distance L from a first plane S at a front point Pf to a front specified plane Sf, and the distance L from a second plane S2 at a rear point Pr to a rear specified plane Sr may be changed as required. For example, another suitable line may be used rather than the center axis when strictly defining a center axis is difficult, such as when aneurysm is present in the blood vessel and the like. Furthermore, the distance L may be temporarily increased so as to collect the aneurysm of the blood vessel between the front point Pf and rear point Pr since the diameter of the tissue is changeable as in the case of an aneurysm in the blood vessel. Moreover, part of the guide line may also be separated from the center axis of the tubular tissue when the guide line is set where a plurality of blood vessels lie astride one another.

(7) The present invention is also applicable to tubular tissue other than a blood vessel 21, such as the trachea, alimentary canal, lymph glands, nerves and the like.

(8) The MIP image G2 may also be prepared by a MIP process using image data (volume data VD) obtained by masking (not displaying) the heart and other organs included in the image data (volume data VD) using a region extraction process or the like. In this case, tubular tissue can be accurately observed because organs near the target tubular tissue can be eliminated, for example the heart can be excluded from coronary observing. Bones may also be masked and excluded from observation.

(9) Rather than a mouse for specifying points, a trackball type pointing device and/or keyboard may be used.

(10) The front specified plane Sf and rear point Pr need not be calculated each time, since the front specified plane Sf and rear point Pr may be determined once and stored in a memory to be read out later. This modification is effective when a user desires to confirm a prior image on display while switching images in the vicinity of the part of interest.

(11) Volume data of four dimensions or more may also be used. For example, an image may be generated from frames formed of four-dimensional volume data having time series information, and a single image may be generated by visualization of movement information from four-dimensional volume data.

(12) A plurality of volume data may also be used in the present invention. For example, a single image (fusion image) may be prepared from a plurality of volume data obtained from a plurality of devices.

The present examples and embodiments are to be considered as illustrative and not restrictive, and the invention is not to be limited to the details given herein, but may be modified within the scope and equivalence of the appended claims.

What is claimed is:

1. A method for generating an image by projecting image data of three or more dimensions in a region of interest on a two-dimensional plane, the method comprising:
setting a guide curve;
setting a reference direction;
displaying the guide curve on a screen of a monitor;
specifying two or more specification points on the guide curve to designate a partial curve of the guide curve;
acquiring a front point located on the partial curve at a frontmost position in the reference direction;
acquiring a rear point located on the partial curve at a rearmost position in the reference direction;
specifying two planes that encapsulate the partial curve viewed from the reference direction based on the front point and the rear point; and
defining the region of interest based on the two specified planes.

2. The method according to claim 1, wherein the guide curve represents a center axis of tubular tissue.

3. The method according to claim 1, wherein the image data of three or more dimensions within the region of interest includes image data of the tubular tissue and image data of organs.

4. The method according to claim 1, further comprising:
generating an image by maximum intensity projection method using image data of three or more dimensions.

5. The method according to claim 1, wherein the image data of three or more dimensions include image data.

6. The method according to claim 1, wherein said specifying two or more specification points on the guide curve includes specifying one point on the guide curve and using the specified point as a reference to specify two or more specification points.

7. The method according to claim 1, further comprising:
resetting a reference direction;
acquiring a new front point located at a frontmost position on the partial curve in the reset reference direction;
acquiring a new rear point located at a rearmost position on the partial curve in the reset reference direction; and
re-specifying two planes based on the new front point and the new rear point and defining the region of interest based on the two re-specified planes.

8. The method according to claim 1, wherein the reference direction is the direction of image projection on a two-dimensional plane.

9. The method according to claim 1, wherein said specifying two planes includes:
generating a first plane perpendicular to the reference direction and intersecting the front point; generating a second plane perpendicular to the reference direction and intersecting the rear point;
generating a front specified plane parallel to the first plane and located frontward by a predetermined distance from the first plane in the reference direction; and
generating a rear specified plane parallel to the second plane and located rearward by a predetermined distance from the second plane in the reference direction;
wherein the front specified plane and the rear specified plane respectively correspond to the two planes encapsulating the partial curve.

10. A computer program device including a computer readable recording medium encoded with a program for projecting, on a two-dimensional plane, image data of three or more dimensions in a region of interest and generating an image by executing either one of independent processing or distributed processing with at least one computer, the program when executed by the at least one computer performing a method comprising:
setting a guide curve;
setting a reference direction;
displaying the guide curve on a screen of a monitor;
specifying two or more specification points on the guide curve to designate a partial curve of the guide curve;
acquiring a front point located on the partial curve at a frontmost position in the reference direction;
acquiring a rear point located on the partial curve at a rearmost position in the reference direction;
specifying two planes that encapsulate the partial curve viewed from the reference direction based on the front point and the rear point; and
defining the region of interest based on the two specified planes.

11. The computer program device according to claim 10, wherein the guide curve represents the center axis of tubular tissue.

12. The computer program device according to claim 10, wherein the image data of three or more dimensions within the region of interest includes image data of the tubular tissue and image data of organs.

13. The computer program device according to claim 10, wherein said method further comprises:
generating an image by a maximum intensity projection method using image data of three or more dimensions.

14. The computer program device according to claim 10, wherein the image data of three or more dimensions include image data of an organ.

15. The computer program device according to claim 10, wherein said specifying two or more specification points on the guide curve includes specifying one point on the guide curve and using the specified point as a reference to specify two or more specification points.

16. The computer program device according to claim 10, wherein the program further comprises:
resetting a reference direction;
acquiring a new front point located at a frontmost position on the partial curve in the reset reference direction;
acquiring a new rear point located at a rearmost position on the partial curve in the reset reference direction; and
re-specifying two planes based on the new front point and the new rear point and defining the region of interest based on the two re-specified planes.

17. The computer program device according to claim 10, wherein the reference direction is the direction of image projection on a two-dimensional plane.

18. The computer program device according to claim 10, wherein said specifying two planes includes:
generating a first plane perpendicular to the reference direction and intersecting the front point;
generating a second plane perpendicular to the reference direction and intersecting the rear point;
generating a front specified plane parallel to the first plane and located frontward by a predetermined distance from the first plane in the reference direction; and
generating a rear specified plane parallel to the second plane and located rearward by a predetermined distance from the second plane in the reference direction;
wherein the front specified plane and the rear specified plane respectively correspond to the two planes encapsulating the partial curve.

19. A device for projecting, on a two-dimensional plane, image data of three or more dimensions in a region of interest and generating an image by executing either one of independent processing or distributed processing with at least one computer, the device comprising:
a means for setting a guide curve;
a means for setting a reference direction;
a means for displaying the guide curve on a screen of a monitor;
a means for specifying two or more specification points on the guide curve to designate a partial curve of the guide curve;
a means for acquiring a front point located on the partial curve at a frontmost position in the reference direction;
a means for acquiring a rear point located on the partial curve at a rearmost position in the reference direction;
a means for specifying two planes that encapsulate the partial curve viewed from the reference direction based on the front point and the rear point; and
a means for defining the region of interest based on the two specified planes.

20. The device according to claim 19, wherein the guide curve represents the center axis of tubular tissue.

21. The device according to claim 19, wherein the image data of three or more dimensions within the region of interest includes image data of the tubular tissue and image data of organs.

22. The device according to claim 19, further comprising:
a means for generating an image by a maximum intensity projection method using image data of three or more dimensions.

23. The device according to claim 19, wherein the image data of three or more dimensions include image data of an organ.

24. The device according to claim 19, wherein said means for specifying two or more specification points on the guide curve includes specifying one point on the guide curve and using the specified point as a reference to specify two or more specification points.

25. The device according to claim 19, wherein when the means for setting a reference direction resets the reference direction:
the means for acquiring a front point acquires a new front point located at a frontmost position on the partial curve in the reset reference direction;
the means for acquiring a rear point acquires a new rear point located at a rearmost position on the partial curve in the reset reference direction; and
the means for specifying two planes re-specifies two planes based on the new front point and the new rear point and defines the region of interest based on the two re-specified planes.

26. The device according to claim 19, wherein the reference direction is the direction of projecting image data of three or more dimensions on a two-dimensional plane.

27. The device according to claim 19, wherein said means for specifying two planes:
generates a first plane perpendicular to the reference direction and intersecting the front point;
generates a second plane perpendicular to the reference direction and intersecting the rear point;
generates a front specified plane parallel to the first plane and located frontward by a predetermined distance from the first plane in the reference direction; and
generates a rear specified plane parallel to the second plane and located rearward by a predetermined distance from the second plane in the reference direction;
wherein the front specified plane and the rear specified plane respectively correspond to the two planes encapsulating the partial curve.
